(19) United States
(12) Patent Application Publication
(10) Pub. No.: US 2024/0185969 A1
(43) Pub. Date: Jun. 6, 2024

(54) SYSTEMS AND METHODS FOR AUTOMATICALLY SELECTING MEDICARE ADMINISTRATIVE CONTRACTORS

(71) Applicant: Change Healthcare Holdings, LLC, Nashville, TN (US)

(72) Inventors: Chrissy Finn, Melrose, MA (US); Neil Davis, Wheat Ridge, CO (US); Rith Peou, Winchester, MA (US); Marissa Sanville, Cherry Hill, NJ (US); Allen Saunders, Philadelphia, PA (US); Christine Yee, Medford, MA (US)

(21) Appl. No.: 18/062,059

(22) Filed: Dec. 6, 2022

(51) Int. Cl. G16H 10/60

(52) U.S. Cl. CPC G16H 10/60

(57) ABSTRACT

In an embodiment, systems and methods for automatically determining a MAC for a medical review request and for automatically selecting the determined MAC in a graphical user interface is provided. A request for a medical review for a medical procedure is provided. Based on information associated with the medical procedure, such as a location of the procedure, a MAC with jurisdiction over the procedure is automatically determined for a user. The determined MAC is then preselected in a graphical user interface and is used to determine an LCD or an NCD that applies to the medical procedure associated with the request for a medical review. A series of questions are presented based in the graphical user interface based on the determined LCD or NCD.

### BACKGROUND

Currently, there are multiple Medicare Administrative Contractors (“MACs”), each responsible for processing Medicare Part A and Part B medical claims for specific jurisdictions in the United States. Each MAC may be further responsible for establishing local coverage determinations (“LCDs”) for their jurisdictions that are policies that explain the requirements for one or more medical procedures to be reimbursed by Medicare. In addition, there also exists national coverage determinations (“NCDs”) for one for more medical procedures.

Typically, when an entity such as a payor or medical provider wants to determine if a particular medical procedure for a patient will be covered by Medicare, the entity must first select the MAC that applies to the medical procedure. The selected MAC and medical procedure are then used to determine the LCDs and NCDs that may apply to the medical procedure. An appropriate questionnaire may then be generated for the entity based on the determined LCDs and NCDs and may be used to provide a medical review for the medical procedure.

One drawback associated with such an approach is that sometimes it may be difficult for the entity to determine which MAC applies to a medical procedure. Each state may be associated with multiple MACs, with each MAC serving a defined geographic region of the state. Thus, an entity must know the exact location in a state where the procedure will be performed to determine the appropriate MAC for the service. In addition, the MAC associated with a geographic region may change requiring the entity to monitor for changes to MACs and their coverage areas.

For entities such as insurance companies, determining the appropriate MAC to select for a medical review may be time consuming but manageable. However, insurance companies are now providing portals through which entities such as medical providers can submit their own medical review requests. Because medical providers are less familiar with MACs and medical reviews, determining the appropriate MAC to select for a medical review may be confusing, which may result in delayed and incorrect requests for medical reviews.

### SUMMARY

In an embodiment, systems and methods for automatically determining a MAC for a medical review request and for automatically selecting the determined MAC in a graphical user interface is provided. A request for a medical review for a medical procedure is provided. Based on information associated with the medical procedure, such as a location of the procedure, a MAC with jurisdiction over the procedure is automatically determined for a user. The determined MAC is then preselected in a graphical user interface and is used to determine an LCD or an NCD that applies to the medical procedure associated with the request for a medical review. A series of questions are presented in the graphical user interface based on the determined LCD or NCD.

In an embodiment, a method is provided. The method includes: receiving a request for a medical review for a medical procedure for a patient by a computing device through a graphical user interface; based on the request for the medical review, automatically determining a Medicare administrative contractor of a plurality of Medicare administrative contractors that covers the request for review of the medical procedure by the computing device; based on the determined Medicare administrator and the medical procedure, determining one or more determinations that apply to the medical review by the computing device, generating a plurality of questions for the medical review based on the determined one or more determinations by the computing device; and presenting the plurality of questions in the graphical user interface.

Embodiments may include some or all of the following features. The method may further include receiving answers to the plurality of questions and determining if one or more requirements of the one or more determinations have been met based on the answers. The one or more determinations may include one or more of national coverage determinations, local coverage determinations, and payor-specific coverage determinations. Automatically determining the Medicare administrative contractor of the plurality of Medicare administrative contractors that covers the request for review of the medical procedure may include determining a location associated with the medical procedure, and automatically determining the Medicare administrative contractor of the plurality of Medicare administrative contractors that covers the request for review of the medical procedure based on the location. The request for review may include a state, a zip code, or an address, and further comprising determining the location based on the state, zip code, or the address. The method may further include: presenting a list of the plurality of Medicare administrative contractors in the graphical user interface; and preselecting the determined Medicare administrative contractor in the list of the plurality of administrative contractors in the graphical user interface. A user may or may not change the preselected determined Medicare administrative contractor based on one or more preferences of a payor associated with the request.

In an embodiment, a system is provided. The system includes at least one computing device; and a computer-readable medium. The computer-readable medium storing computer-executable instructions that when executed by the at least one computing device cause the system to: render a graphical user interface; receive a request for a medical review for a medical procedure for a patient through the graphical user interface, wherein the request is associated with a payor; retrieve preferences associated with the payor; based on the request for the medical review and the preferences, automatically determine a Medicare administrative contractor of a plurality of Medicare administrative contractors that covers the request for review of the medical procedure by the computing device; based on the determined Medicare administrative contractor, the medical procedure, and the preferences, determine one or more determinations that apply to the medical procedure by the computing device; generate a plurality of questions for the medical review based on the determined one or more determinations; and present the plurality of questions in the graphical user interface.

Embodiments may include some or all of the following features. The graphical user interface may be part of a portal application provided by the payor and the request for medical review is received from a medical service provider through the portal application. The one or more determinations may include one or more of a national coverage determination, a local coverage determination, and a payor-specific coverage determination. Automatically determining the Medicare administrative contractor of the plurality of Medicare administrative contractors that covers the request for review of the medical procedure may include determining a location associated with the medical procedure, and automatically determining the Medicare administrative contractor of the plurality of Medicare administrative contractors that covers the request for review of the medical procedure based on the location. The request for review may include a state, a zip code, or an address, and further comprising determining the location based on the state, zip code, or the address. The system may further include computer-executable instructions that when executed by the at least one computing device cause the system to: present a list of the plurality of Medicare administrative contractors in the graphical user interface; and preselect the determined Medicare administrative contractor in the list of the plurality of administrative contractors in the graphical user interface. A user may or may not change the preselected determined Medicare administrative contractor based on the preferences.

The systems and methods described herein provide the following advantages. Because the MAC for a medical review is determined automatically and without input from the medical provider, the speed at which medical reviews can be created and submitted is greatly increased. Moreover, automatically determining the MAC results in a decrease in incorrect and rejected medical review requests for medical procedures.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate systems and methods for automatically selecting Medicare administrative contractors for a medical review. Together with the description, the figures further serve to explain the principles of the systems and methods described herein and thereby enable a person skilled in the pertinent art to make and use the systems and methods.

FIG. 1 is an example environment for handling requests for medical review;

FIG. 2 is an illustration of a method for automatically determining a MAC for a medical review and for performing the medical review according to one or more determinations;

FIG. 3 is an illustration of a method for or performing the medical review according to payor preferences; and

FIG. 4 shows an example computing environment in which example embodiments and aspects may be implemented.

### DETAILED DESCRIPTION

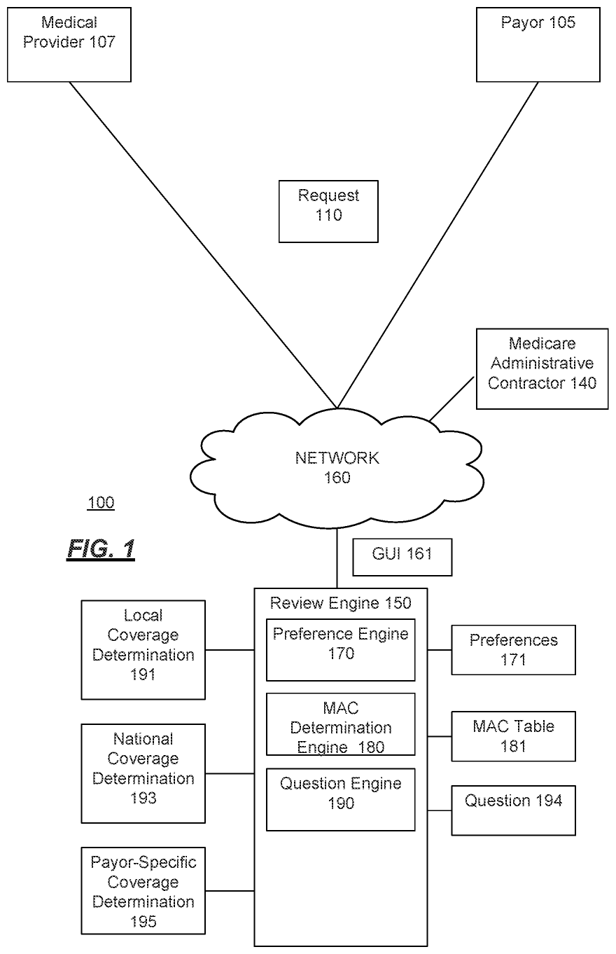

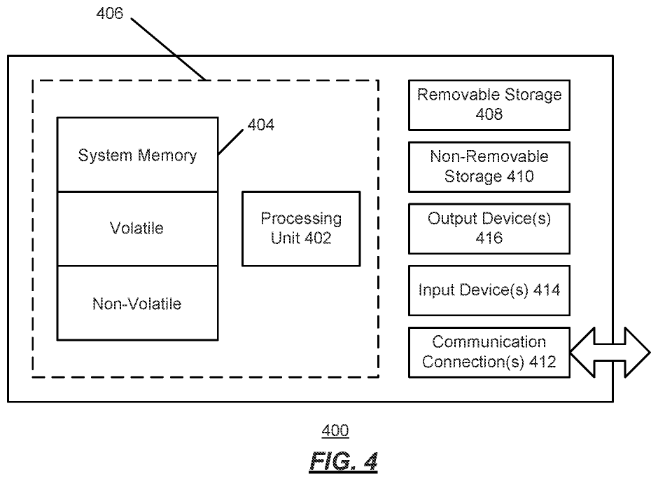

FIG. 1 is an example environment 100 for handling requests 110 for medical review. As shown, the environment 100 may include one or more medical providers 107, one or more payors 105, one or more Medicare administrative contractors 140, and a review engine 150 in communication through a network 160. The network 160 may include a combination of private networks (e.g., LANs) and public networks (e.g., the Internet). Each of the one or more medical providers 107, the one or more payors 105, the one or more Medicare administrative contractors 140, and the review engine 150 may use, or may be partially implemented by, one or more general purpose computing devices such as the computing device 400 illustrated in FIG. 4.

As used herein a medical review may be a decision as to whether a particular patient qualifies for reimbursement for an associated medical procedure based on one or more rules or guidelines associated with the medical procedure. These rules are known as determinations.

Generally, the determinations that apply to a medical procedure may include local coverage determinations 191 that may be specific to a geographic jurisdiction and may be set by one of a plurality of Medicare administrative contractors 140, national coverage determinations 193 that are set by the Federal government and may apply to all jurisdictions, and payor-specific coverage determinations 195 that may be set by a particular payor 105.

Typically, a medical provider 107 (or a payor 105 on behalf of a medical provider 107) submits a request 110 for medical review to a reviewer application for a medical procedure that the medical provider 107 would like to perform on a patient. The request 110 may include information such as an identifier of the patient, demographic information about the patient (e.g., age and sex), a date when the procedure is to be performed, and a location of where the procedure will occur. Other information may be included.

The reviewer application, upon receiving the request 110, first prompts the submitter of the request 110 to select a Medicare administrative contractor 140 that has jurisdiction over Medicare claims where the medical service associated with the request 110 is to be performed. As described above, the United States may be divided into a plurality of geographic regions with each region awarded to one of a plurality of Medicare administrative contractors 140. Each Medicare administrative contractor 140 has authority to create its own local coverage determination 191 for each medical procedure.

Accordingly, before the reviewer application can determine what questions 194 to ask the submitter of the request 110 to evaluate the appropriateness of the procedure associated with the request 110, the reviewer application must first know which Medicare administrative contractor 140 has jurisdiction. However, as noted above, persons who submit a request 110 may not know which Medicare administrative contractor 140 applies to the request and may not be able to correctly determine the Medicare administrative contractor 140 for the reviewing application.

Accordingly, to solve these and other problems, the environment 100 includes a review engine 150 that automatically determines the Medicare administrative contractor 140 that applies to request 110 for medical review without feedback or input from a user. As shown, the review engine 150 includes several components including, but not limited to, a preferences engine 170, a MAC determination engine 180, and a questions engine 190. More or fewer components may be supported. Each component of the review engine 150 may be implemented together or separately using one or more general purpose computing devices such as the computing device 400 illustrated with respect to FIG. 4.

The payors 105 and medical providers 107 may connect to and interact with the review engine 150 using a graphical user interface 161 provided by the review engine 150. Alternatively, or additionally, the payor 105 may provide its own GUI 161 that interacts with the review engine 150 through a software API provided by the review engine 150. Where GUI 161 is provided by the payor 105, it may be a portal application through which the payor 105 and associated medical providers 107 may connect to the review engine 150.

A medical provider 107 or payor 105 may provide a request 110 for a medical review of a proposed medical procedure. The request 110 may include a variety of information such as an identifier of the medical procedure, demographic information about the patient, a location where the procedure will be performed, and a date when the medical procedure will be performed. Other information may be included.

When a request 110 is received, the preferences engine 170 may determine if there are any preferences 171 associated with the payor 105 that submitted the request 110 or whose portal or GUI 161 is being used by a medical provider 107 to submit the request. Preferences 171 may be set or provided by the payor 105 and may include preferences 171 with regards to the types of determinations that should be used to perform medical reviews for requests 110.

For example, the preferences 171 may indicate that a payor 105 prefers to use national coverage determinations 193 instead of local coverage determinations 191 when available, prefers to use its own payor-specific determinations 195 when available, or prefers to use a combination of the determinations 191, 193, and 195. Depending on the embodiment, the preferences 171 for a payor 105 may broadly apply to all medical procedures or may be specific to different types of medical procedures. Additionally, as will be described further below, the preferences 171 may specify whether the selected determination may be changed or overridden in the GUI 161.

Where the preferences 171 indicate that local coverage determination 191 should be used for the medical review, the MAC determination engine 180 may determine the MAC 140 with jurisdiction over the medical procedure indicated by the request 110. The MAC determination engine 180 may determine the MAC 140 based on the location where the medical procedure will be performed. In some embodiments, the MAC determination engine 180 may look up the location in a MAC table 181 that maps locations to MACs 140. An example location that may be used with the MAC table 181 may include the state where the medical procedure will be performed, an address of the clinic or hospital where the medical procedure will be performed, and a zip code of where the medical procedure will be performed. The MAC table 181 may be generated by the MAC determination engine 180 based on information provided about each MAC 140 and their associated jurisdictions by the Federal government.

In some embodiment, to determine the MAC 140 with jurisdiction over the medical procedure, the MAC determination engine 180 may also use a date associated with the medical procedure. As may be appreciated, the jurisdictions associated with each MAC 140 may change or may be reassigned periodically according to a schedule. Therefore, the date of the medical procedure may be needed to determine which MAC 140 has jurisdiction over the medical procedure.

After determining the MAC 140 that has jurisdiction over the medical service associated with the request, the MAC determination engine 180 may display the determined MAC 140 in the GUI 161 to the provider 107 that provided the request 110. In some embodiments, the MAC 140 may be displayed along with other MACs 140. The displayed MACs 140 may be MACs 140 with jurisdictions that are close to the location associated with the request 110. Depending on the preferences 171 for the payor 105, the provider 107 may or may not be able to select a different MAC 140 from among the displayed MACs 140 in the GUI 161.

After the MAC 140 is determined by the MAC determination engine 180, the question engine 190 may begin generating questions 194 for the medical review. In some embodiments, the question engine 190 may determine the local coverage determination 191 that applies to the medical procedure of the request 110 for the determined MAC 140. The question engine 190 may then select or generate one or more questions 194 based on the requirements of the local coverage determination 191.

For example, the local coverage determination 191 for a medical procedure such as a knee replacement may include several requirements such as advanced knee disease, a history of unsuccessful conservative treatments, pain, or structural abnormalities. Accordingly, the question engine 190 may generate questions 194 for the medical review such as: “Does the patient have an advanced knee disease?”, “Please select an advanced knee disease of the patient from the following list”, “Please list any prior treatments that that the patient may have had”. Other questions may be supported. Depending on the embodiment, the questions 194 may be generated or may be selected based on the local coverage determination 191. For example, there may be a database of pre-generated questions 194 with each question 194 tagged or otherwise associated with a particular medical procedure and MAC 140.

The question engine 190 may provide the generated questions 194 to the medical provider 107 through the GUI 161. Depending on how some of the questions 194 are answered, the question engine 190 may select or generate follow up questions 194 that may be displayed in the GUI 161. Once the questions 194 have been completed, the question engine 190 may determine whether the requirements of the local coverage determination 191 have been met and may notify medical provider 107 in the GUI 161.

In addition to, or instead of, the local coverage determination 191, the question engine 190 may also consider one or both of the national coverage determination 193 or payor-specific coverage determination 195 when generating the questions 194. In some embodiments, the preferences 171 may specify that the medical review should have questions 194 based on the local coverage determination 191 or the payor-specific coverage determination 195, or may specify that the medical review should have questions 194 based on the national coverage determination 193 or the payor-specific coverage determination 195 instead of the local coverage determination 191. In other embodiments, the question engine 190 may consider both of the national coverage determination 193 or payor-specific coverage determination 195 for the questions 194 when there is no local coverage determination 191 for the medical procedure associated with the request 110 for the medical review. In some cases, there may be no local coverage determination 191 associated with a particular medical procedure, and the question engine 190 may use the national coverage determination 193 or the payor-specific coverage determination 195.

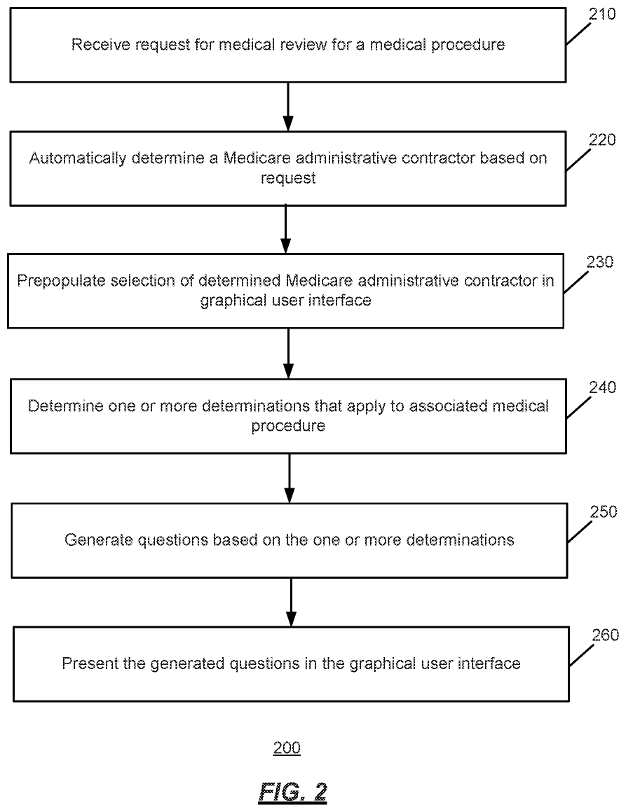

FIG. 2 is an illustration of a method for automatically determining a MAC 140 for a medical review and for performing the medical review according to a one or more determinations.

At 210, a request for a medical review of a medical procedure is received. The request 110 may be received by the review engine 150 from a medical provider 107. The request may be received through a GUI 161 provided by the review engine 150. The medical provider 107 may be a provider of medical services such as a hospital, a clinic, or a doctor's office. The request 110 for medical review may include information such as a location and date of the medical procedure and demographic information about a patient that will receive the medical procedure from the medical provider 107. The patient may be associated with the payor 105 (e.g., an insurance company) and may receive medical insurance from the payor 105.

At 220, a Medicare administrative contractor is determined based on the request. The MAC determination engine 180 may determine the MAC 140 with jurisdiction over the medical procedure referenced in the request 110 based on a location associated with the medical procedure. In some embodiments, the location may include a zip code or address of where the medical procedure will be performed and the MAC determination engine 180 may determine the MAC 140 using the location and a MAC table 181 that maps locations to MACs 140.

At 230, a selection of the determined Medicare administrative contractor is prepopulated in the graphical user interface. The selection of the determined MAC 140 may be prepopulated in the GUI 161 by the MAC determination engine 180. In some embodiments, other MACs 140 may also be prepopulated in the GUI 161, however these MACs 140 may not be preselected. Depending on the embodiment, the medical provider 107 may or may not be able to select a different MAC 140 from the MAC 140 preselected in the GUI 161 depending on preferences 171 associated with the payor 105, for example.

At 240, a determination that applies to the medical procedure associated with the request is determined. The determination may be one or more of a local coverage determination 191, a national coverage determination 193, or a payor-specific determination 195. The applicable determination or determinations may be determined by the question engine 190 based on the determined MAC 140 and one or more payor preferences 171.

At 250, one or more questions are generated based on the one or more determinations. The one or more questions 194 may be generated by the question engine 190. Where multiple determinations are determined, the question engine 190 may generate the one or more questions based on each of the determinations. In some embodiments, the questions 194 may be predetermined and tagged based on the MAC 140 and/or medical procedure that they correspond to.

At 260, the generated questions are presented. The questions 194 may be presented in the GUI 161 to the medical provider 107 that provided the request 110 as part of the medical review.

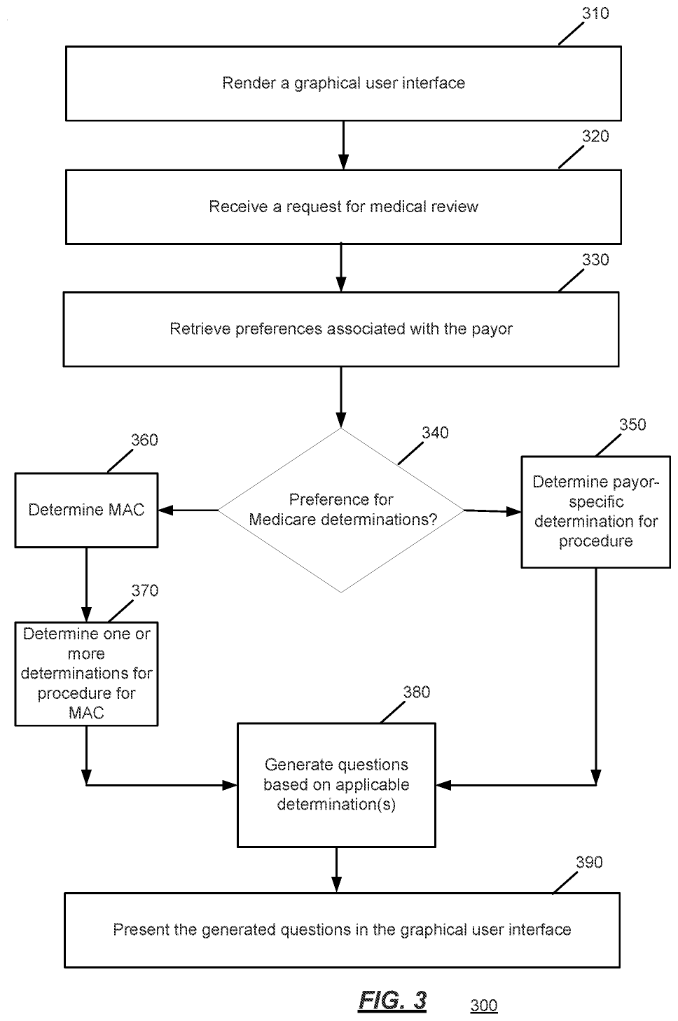

FIG. 3 is an illustration of a method for or performing the medical review according to payor preferences The method 300 may be implemented by the review engine 150.

At 310, a graphical user interface is rendered. The GUI 161 may be rendered by the review engine 150. Alternatively, the GUI 161 may be a portal application rendered by the payor 105 for use by a medical provider 107 using an API provided by the review engine 150.

At 320, a request for a medical review of a medical procedure is received. The request 110 may be received by the review engine 150 from the medical provider 107. The request 110 for medical review may include information such as a location and date of the medical procedure and demographic information about a patient that will receive the medical procedure from the medical provider 107. The patient may be associated with the payor 105 and may receive medical insurance from the payor 105.

At 330, preferences associated with the payor are retrieved. The preferences 171 may be retrieved by the preference engine 170.

At 340, whether the payor has a preference for using Medicare determinations for performing medical reviews is determined. In some embodiments, the preferences 171 may indicate whether the payor 105 prefers to use local coverage determinations 191, national coverage determinations 193, or payor-specific coverage determinations 195. If the payor 105 prefers payor-specific coverage determinations 195 the method 300 may continue at 350. Else the method 300 may continue 360.

At 350, a payor-specific determination corresponding to the medical procedure associated with the request 110 is determined. The determination 195 may be determined by the question engine 190. In the event that no payor-specific determination 195 corresponding to the medical procedure exists, an error may be generated. Else, the method 300 may continue at 380.

At 360, a Medicare administrative contractor is determined. The MAC determination engine 180 may determine the MAC 140 with jurisdiction over the medical procedure referenced in the request 110 based on a location associated with the medical procedure.

At 370, one or more determinations for the Medicare administrative contractor that applies to the medical procedure associated with the request are determined. The determinations, which may include local coverage determinations 191, national coverage determinations 193 and/or payer-specific determinations 195, may be determined by the question engine 190.

At 380, one or more questions are generated. The one or more questions 194 may be generated by the question engine 190 from one or more of the local coverage determinations 191, the national coverage determination 193, or the payor-specific determination 195. In some embodiments, some of the questions 194 may have been pre-generated and may be selected by the question engine 190 based on tags or other indications of the determinations 191, 193, and 195 that the questions relate to.

At 390, the generated questions are presented. The questions 194 may be presented in the GUI 161 to the payor 105 and/or medical provider 107 that provided the request 110.

FIG. 4 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 4, an example system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 4 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 4 by removable storage 408 and non-removable storage 410.

Computing device 400 typically includes a variety of computer readable media. Computer readable media can be any available media that can be accessed by the device 400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media may be part of computing device 400.

Computing device 400 may contain communication connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
receiving a request for a medical review for a medical procedure for a patient by a computing device through a graphical user interface;
based on the request for the medical review, automatically determining a Medicare administrative contractor of a plurality of Medicare administrative contractors that covers the request for review of the medical procedure by the computing device;
based on the determined Medicare administrator and the medical procedure, determining one or more determinations that apply to the medical review by the computing device,
generating a plurality of questions for the medical review based on the determined one or more determinations by the computing device; and
presenting the plurality of questions in the graphical user interface.

2. The method of claim 1, further comprising receiving answers to the plurality of questions and determining if one or more requirements of the one or more determinations have been met based on the answers.

3. The method of claim 1, wherein the one or more determinations comprise one or more of national coverage determinations, local coverage determinations, and payor-specific coverage determinations.

4. The method of claim 1, wherein automatically determining the Medicare administrative contractor of the plurality of Medicare administrative contractors that covers the request for review of the medical procedure comprises determining a location associated with the medical procedure, and automatically determining the Medicare administrative contractor of the plurality of Medicare administrative contractors that covers the request for review of the medical procedure based on the location.

5. The method of claim 1, wherein the request for review includes a state, a zip code, or an address, and further comprising determining the location based on the state, zip code, or the address.

6. The method of claim 1, further comprising:
presenting a list of the plurality of Medicare administrative contractors in the graphical user interface; and
preselecting the determined Medicare administrative contractor in the list of the plurality of administrative contractors in the graphical user interface.

7. The method of claim 1, wherein a user may or may not change the preselected determined Medicare administrative contractor based on one or more preferences of a payor associated with the request.

8. A system comprising:
at least one computing device; and
a computer-readable medium storing computer-executable instructions that when executed by the at least one computing device cause the system to:
render a graphical user interface;
receive a request for a medical review for a medical procedure for a patient through the graphical user interface, wherein the request is associated with a payor;
retrieve preferences associated with the payor;
based on the request for the medical review and the preferences, automatically determine a Medicare administrative contractor of a plurality of Medicare administrative contractors that covers the request for review of the medical procedure by the computing device;
based on the determined Medicare administrative contractor, the medical procedure, and the preferences, determine one or more determinations that apply to the medical procedure by the computing device;
generate a plurality of questions for the medical review based on the determined one or more determinations; and
present the plurality of questions in the graphical user interface.

9. The system of claim 8, wherein the graphical user interface is part of a portal application provided by the payor and the request for medical review is received from a medical service provider through the portal application.

10. The system of claim 8, wherein the one or more determinations comprise one or more of a national coverage determination, a local coverage determination, and a payor-specific coverage determination.

11. The system of claim 8, wherein automatically determining the Medicare administrative contractor of the plurality of Medicare administrative contractors that covers the request for review of the medical procedure comprises determining a location associated with the medical procedure, and automatically determining the Medicare administrative contractor of the plurality of Medicare administrative contractors that covers the request for review of the medical procedure based on the location.

12. The system of claim 11, wherein the request for review includes a state, a zip code, or an address, and further comprising determining the location based on the state, zip code, or the address.

13. The system of claim 8, further comprising computer-executable instructions that when executed by the at least one computing device cause the system to:
present a list of the plurality of Medicare administrative contractors in the graphical user interface; and
preselect the determined Medicare administrative contractor in the list of the plurality of administrative contractors in the graphical user interface.

14. The system of claim 8, wherein a user may or may not change the preselected determined Medicare administrative contractor based on the preferences.

15. A computer-readable medium storing computer-executable instructions that when executed by the at least one computing device cause the system to:
receive a request for a medical review for a medical procedure for a patient through a graphical user interface;
based on the request for the medical review, automatically determine a Medicare administrative contractor of a plurality of Medicare administrative contractors that covers the request for review of the medical procedure;
based on the determined Medicare administrative contractor and the medical procedure, determine one or more determinations that apply to the medical review by the computing device;
generate a plurality of questions for the medical review based on the determined one or more determinations by the computing device; and
present the plurality of questions in the graphical user interface.

16. The computer-readable medium of claim 15, further comprising receiving answers to the plurality of questions and determining if one or more requirements of the one or more determinations have been met.

17. The computer-readable medium of claim 15, wherein the one or more determinations comprise one or more of national coverage determinations, local coverage determinations, and payor-specific coverage determinations.

18. The computer-readable medium of claim 15, wherein automatically determining the Medicare administrative contractor of the plurality of Medicare administrative contractors that covers the request for review of the medical procedure comprises determining a location associated with the medical procedure, and automatically determining the Medicare administrative contractor of the plurality of Medicare administrative contractors that covers the request for review of the medical procedure based on the location.

19. The computer-readable medium of claim 18, wherein the request for review includes a state, a zip code, or an address, and further comprising determining the location based on the state, zip code, or the address.

20. The computer-readable medium of claim 18, further comprising:
presenting a list of the plurality of Medicare administrative contractors in the graphical user interface; and
preselecting the determined Medicare administrative contractor in the list of the plurality of administrative contractors in the graphical user interface.
